(19) United States
(12) Patent Application Publication
(10) Pub. No.: US 2015/0366703 A1
(43) Pub. Date: Dec. 24, 2015

(54) ALTERNATING HOT AND COLD THERAPY APPARATUS

(71) Applicant: Xing Du, Newport Beach, CA (US)

(72) Inventor: Xing Du, Newport Beach, CA (US)

(21) Appl. No.: 14/307,535

(22) Filed: Jun. 18, 2014

(51) Int. Cl. A61F 7/08; A61F 7/00

(52) U.S. Cl. CPC A61F 7/08; A61F 7/007; A61F 2007/0054; A61F 2007/0056; A61F 2007/0093; A61F 2007/0095

(57) ABSTRACT

An alternating hot and cold therapy apparatus that applies alternating heat and cold to a pain affected area. The apparatus includes a housing, a therapy pad, and a flexible hose connecting the housing to the therapy pad. The housing contains a fluid reservoir and hydraulic pump in fluid communication with the reservoir, a fluid line connected to the pump, a programmable controller operably connected to the pump, a timer for providing time data to the controller, a cooling means to cool the fluid, a power source to supply power to the controller and pump, and a conductor from the power source to the therapy pad. The therapy pad includes a plurality of thermoelectric chips that generate a temperature differential in response to an electric current. The flexible hose carries the fluid line and conductor to the therapy pad where the fluid line and conductor wind through the therapy pad, contacting each of the thermoelectric chips. In cooling mode, an electric current flows through the thermoelectric chips causing one side to cool and the other to warm. The apparatus circulates fluid through the therapy pad to dissipate heat from the warm side. In heating mode an electric current is applied to the thermoelectric chips, but circulating fluid is turned off, allowing heat to build up in the chips.

### FIELD OF THE INVENTION

The present invention relates generally to heat and cold therapy. Specifically, the invention relates to an apparatus for providing alternating hot and cold therapy to a pain affected area of the body.

### BACKGROUND OF THE INVENTION

Pain caused by arthritis, muscle ache, joint inflammation, repetitive motion pain, strains, sprains, post-surgical pain, and many other types of pain can be treated with various forms of therapeutic intervention. Some of these interventions include topical treatments, massage, muscle stimulation, acupuncture, medications, hydrotherapy, and hot/cold compresses. The effectiveness of these treatments often varies from case to case depending on the particular type of pain and its cause.

In the case of hot/cold treatments various methods and devices may be used to transmit heat and/or cold to an affected area. Some of these include chemicals that turn hot or cold based on exothermic or endothermic reactions, electrical devices that generate heat or cold, hot/cold packs that use fluids to store and transmit heat/cold, or topical treatments that provide a hot or cold sensation upon contact with skin.

In some cases, however, an effective treatment regimen calls for rapid application of alternating heat and cold to an affected area. Existing methods and devices are generally not very effective in achieving rapid alternating temperature changes from heat to cold, but this is sometimes exactly what a patient needs to feel relief from the pain they are suffering. Furthermore, trying to switch back and forth between a heat producing device and a cold producing device in rapid succession is cumbersome and generally not practical. Moreover, hot and cold packs can quickly lose their capacity to produce heat and cold and may require substantial preparation time before they can be used. There is therefore a need for a therapeutic device that is capable of applying rapid alternating heat and cold to an affected area for pain relief in an efficient, practical manner.

### SUMMARY OF THE INVENTION

Accordingly, it is an object of present invention to provide a method and apparatus for providing rapid alternating therapeutic heat and cold to an area of the body to help relieve pain and, if possible, improve healing.

According to an embodiment of the present invention, an alternating heating and cooling apparatus for treating pain is provided. The apparatus includes a housing having a fluid reservoir, a hydraulic pump, a controller, and a power supply. The housing is connected to a therapy pad via a flexible hose containing at least one fluid line which is connected to the pump. The fluid line transmits and circulates fluid from the reservoir into the therapy pad when the pump is operating and then returns it to the reservoir. The therapy pad also includes a plurality of thermoelectric chips with resistance wire embedded therein disposed in series along the fluid line. The tube connecting the housing to the therapy pad may also contain a conductor that carries electricity from a power supply to the thermoelectric chips.

According to an embodiment of the present invention, the thermoelectric chips may assume a cooling state (cooling mode) or heat generating state (heating mode). During cooling mode the thermoelectric chips receives an electric current causing a temperature differential between the top and bottom sides of the chip, i.e. one side heats up while the other side cools. The hydraulic pump sends fluid from the housing through the fluid line that winds through the therapy pad. The fluid line is connected to the thermoelectric chips and serves to cool the hot side of the chips. Thus, when the therapy pad is applied to a user's skin, the plurality of thermoelectric chips provide a cooling sensation.

During heating mode, the hydraulic pump is turned off and a reversed electric current is applied to the thermoelectric chips or electric heating resistance wire embedded in the thermoelectric chips. The reversal of electric current causes the cool side of the thermoelectric chips to become the hot side. In heating mode the fluid line or air is not used to dissipate the heat created on the chip. The transition from cooling mode to heating mode is therefore largely a result of turning the hydraulic pump on and off, which allows the therapy pad to quickly change from heating to cooling mode and vice versa.

According to an embodiment of the present invention, a controller is configured to activate and deactivate cooling mode and heating mode. When cooling mode is activated, the controller turns on the pump and maintains electrical power to the thermoelectric chips. In cooling mode, the temperature of the thermoelectric chips is lowered by running current through the chips and circulating fluid through the therapy pad or using air to dissipate heat from the chips. In heating mode, the controller turns off the pump and maintains reversed electrical current to the chips or to the electric heating resistance wire embedded in the thermoelectric chips. Heat is allowed to build up on the chips causing the therapy pad to radiate heat.

One or more temperature sensors in communication with the controller detect whether the therapy pad is too hot or too cold. If the temperature reaches a predetermined upper limit, the controller automatically shuts off the power so that the thermoelectric chips no longer receive an electrical charge and stop heating up. The controller may also activate cooling mode by turning on the pump, so that cooling fluid circulates through the therapy pad and lowers the temperature. Once the temperature is back within a predetermined normal range, the controller may deactivate cooling mode and optionally activate heating mode.

Similarly, if the temperature of the therapy pad reaches a predetermined lower limit, the controller may automatically shut off the pump. The controller may also activate heating mode by turning on the power, so that electricity is transmitted to the thermoelectric chips, causing them to heat up. Once the temperature is back within a predetermined normal range, the controller may deactivate the heating mode and optionally activate the cooling mode.

According to an embodiment of the present invention, during normal use of the apparatus, the controller automatically activates heating mode and cooling mode, so as to provide alternating heating and cooling to an area in contact with the therapy pad. Alternatively, a user can manually switch between heating mode and cooling mode as desired.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

FIG. 1 is an illustration of an alternating heat and cold therapy apparatus according to an embodiment of the invention.

FIG. 2 is a front elevation view of an alternate embodiment of the invention, which uses air instead of fluid to cool the hot side of the thermoelectric chips in cooling mode.

FIG. 3 is an internal schematic view of an alternating heat and cold therapy apparatus according to an embodiment of the invention.

FIG. 4 is an environmental perspective view showing the alternating heat and cold therapy apparatus in use.

FIG. 5 is a schematic view of the therapy pad portion of the alternating heat and cold therapy apparatus.

### DETAILED DESCRIPTION OF THE INVENTION

The present invention relates generally to hot and cold treatment for pain. Specifically, the invention relates to an apparatus and method for applying alternating heat and cold to an area of the body to help relieve pain and improve healing.

According to an embodiment of the present invention, an apparatus for providing alternating heat and cold to an area of the body is disclosed herein. The apparatus includes a housing having a fluid reservoir, a hydraulic pump, a controller, and a power supply. A therapy pad is connected to the housing via a flexible hose, wherein the hose contains at least one fluid line connected to the hydraulic pump. The fluid line travels through the hose connecting the housing to the therapy pad, winds through the therapy pad, and returns to the housing through the hose. Although the fluid line is enclosed inside the hose, one of ordinary skill in the art will appreciate that the fluid line need not be enclosed inside a hose.

In a preferred embodiment, the therapy pad includes a plurality of thermoelectric chips positioned in series along the fluid line inside the therapy pad. The terms “thermoelectric chip” and “thermo semiconductor” shall be regarded as equivalent terms throughout this application. The thermoelectric chips may be made with alternating n-type and p-type semi-conductors which are placed thermally in parallel to each other and electrically in series and then sandwiched between thermally conducting plates, such as ceramic. When a DC current flows through the chip, it brings heat from one side to the other, so that one side gets cooler while the other gets hotter. The “hot” side is attached to a heat sink, such as the fluid line described herein, which carries heat away from the hot surface of the thermoelectric chip during cooling mode. The number of thermoelectric chips inside the therapy pad may vary, including having only one thermoelectric chip, or a plurality of thermoelectric chips. Furthermore, the thermoelectric chips may be arranged in a variety of ways inside the therapy pad, and embodiments of the present invention are contemplated with any appropriate arrangement of thermoelectric chips.

In a preferred embodiment, the thermoelectric chips may assume a cooling state or heat generating state. In a cooling state, the thermoelectric chips provide a cooling sensation through the therapy pad when the therapy pad is in contact with the user's skin. In a heat generating state, the thermoelectric chips release heat which is applied to the user's skin when the therapy pad is in contact with the user's skin.

According to an embodiment of the present invention, the fluid line inside the therapy pad circulates cooled fluid throughout the therapy pad in close proximity to the thermoelectric chips. For example the fluid line may be positioned to pass over the thermoelectric chips, or through the thermoelectric chips. In a preferred embodiment, the fluid line is directly connected to each thermoelectric chip. After circulating through the therapy pad, the fluid returns to the reservoir inside the housing. The fluid inside the tube draws heat from the thermoelectric chips and carries it away from the therapy pad, back through the line, and into the reservoir.

According to an embodiment of the present invention, the tube connecting the housing to the therapy pad may also contain a conductor such as electrical wire that provides electric current to the thermoelectric chips. When the thermoelectric chips receive the current it causes one side of the chip to heat up and the other side to cool. Reversing the current switches the cooling side and warming side, i.e. the cool side becomes the hot side, and the hot side becomes the cools side. Reversing the current can thus be used to switch a side from warm to cool or vise versa. For example, one side of the therapy pad can alternate between warming and cooling, while the opposite side is kept relatively neutral by applying the fluid line heat sink (or air cooled heat sink) to that side. In other words, the controlled transmission of fluid and/or current may be used to regulate the temperature of the thermoelectric chips.

The surface of the thermoelectric chips may also include electric heating resistance wire. This allows electrical energy transmitted to the thermoelectric chips to be converted to heat. In heating mode, this feature helps generate heat in the chips, and prolong their life.

As explained herein alternating heating and cooling apparatus of the present invention has a heating mode and a cooling mode. In cooling mode the thermoelectric chips' temperature can drop below ambient temperature by up to 20° C. (36° F.). In cooling mode the chips feel cool through the therapy pad and absorb heat from a user's skin. As stated earlier, heat is transferred from the cool side of the chip to the opposite side, but that heat is dissipated by the fluid line connected to the chips or by air directly. The hydraulic pump operates to send fluid from the reservoir into the fluid line circulating inside the therapy pad. The fluid line connected to each chip acts as a heat sink for the chip, i.e. heat from the warm side of the chip is dissipated. In a preferred embodiment, the fluid line is connected to the thermoelectric chips to draw heat away from the thermoelectric chips and carry it back to the reservoir. However, one of ordinary skill in the art will appreciate that any type of heat sink may be used to dissipate heat from the thermoelectric chips, including heat sinks that utilize air, liquid, refrigerant, oil, thermal resistant material, thermal conductive material, or any mechanism that employs Fourier's law.

In heating mode, the thermoelectric chips receive an electrical charge from the conductor that heats up one side of the chips and puts them in a heat generating state. In heating mode, the hydraulic pump is turned off, so that heat can be retained in the thermoelectric chips and radiate from the therapy pad. The transition from cooling to heat generating is thus very quick since the circulating fluid quickly cools the chips in heating mode, while the lack of circulating fluid (i.e. hydraulic pump is turned off) allows heat to quickly build up on the warm side of the chips. The terms “hot” side and “warm” side shall be regarded as equivalent terms throughout this application.

According to an embodiment of the present invention, the controller is configured to activate and deactivate the cooling mode and heating mode according to a preset program and/or based on the temperature of the therapy pad or another component. The controller is configured to receive temperature data from one or more temperature sensors. The temperature sensors may be located in the therapy pad, the connecting hose between the housing and therapy pad, the reservoir, or in some other part of the apparatus. One of ordinary skill in the art will appreciate that there are many possible locations in the apparatus to place a sensor, and embodiments of the present invention are contemplated for use with any appropriate placement of sensors. For example, a sensor in the hose may acquire temperature data for the fluid in the fluid line. If the temperature readings provided by the sensors fall outside a predetermined range, the controller can activate or deactivate the heating/cooling mode until the temperature is back in the predetermined range (i.e. normal range). For instance, if the temperature of the therapy pad rises above a predetermined maximum, the controller can turn off the electrical power to the thermoelectric chips until they cool. In addition, the controller can activate the pump to send cooling fluid through the fluid line to dissipate heat from the thermoelectric chips. Once the temperature falls back within the normal range, the pump can be turned off and electrical power can optionally be turned back on.

Conversely, if the temperature of the therapy pad falls below a predetermined minimum, the controller can turn off the hydraulic pump. Once the temperature of the therapy pad rises back within normal parameters the pump can optionally be turned back on.

In addition, the controller may be operated manually by a user instead of a preset program. A user can thus manually switch between heating mode and cooling mode. However, the controller is able to override either mode if the temperature of the therapy pad is outside of a predetermined normal range. Further, an emergency shut off may be activated if temperatures reach dangerous levels.

According to an embodiment of the present invention, the alternating heating and cooling apparatus may include a means of cooling the fluid in the reservoir. The cooling means may include a fan, condenser, heat exchanger, or heat pump. In addition, the fluid circulating in the system may be water, antifreeze (i.e. glycol), water-antifreeze mixture, or other coolant. One of ordinary skill in the art will appreciate that there are many ways of maintaining the fluid stored in the reservoir at a cool temperature.

According to an embodiment of the present invention, the heating and cooling modes of the apparatus may be controlled manually, such as with a button, switch, remote control, or a computing device programmed to interface with the controller software through an API (Application Programming Interface). As mentioned previously, the controller may be configured to override manual input or a program for safety reasons. For example, if the temperature sensors report abnormally high/low temperatures or if there are other potential hazards detected, such as electrical faults, exposed wiring, or other damage to the apparatus, the controller may initiate an automatic shut-off of one or more components.

According to an embodiment of the invention, the controller may be programmed to provide heat, cold, or alternating heat and cold in timed intervals or sequences. For example, a specified sequence, pattern and duration of heat and cold may be programmed into the controller. In addition, the controller may be programmed to maintain a particular temperature for the therapy pad for a set interval. The temperature and/or interval can be changed in response to manual input or a particular program.

According to an embodiment of the present invention, the power supply provides electrical power to the pump, controller, and thermoelectric chips. The power supply may be a battery, an A/C adapter that draws power from an outlet, a power cord plugged into an outlet, solar power, a small generator, or some other appropriate power supply.

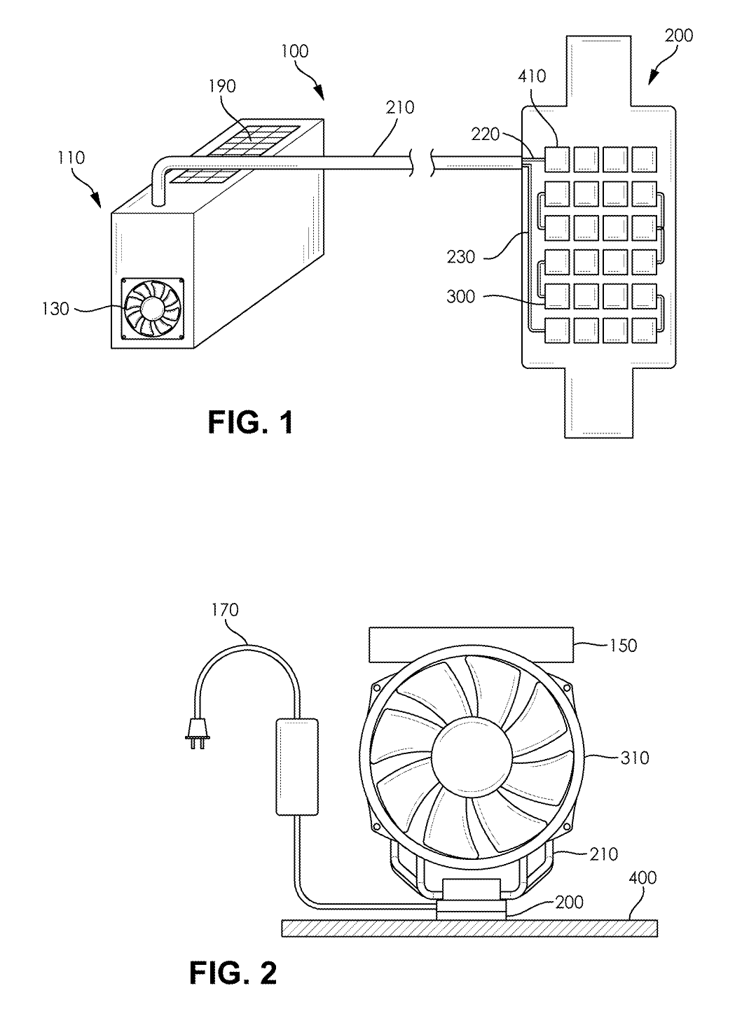

Turning now to FIG. 1, an alternating heat and cold apparatus 100 is illustrated. The apparatus includes a housing 110, a therapy pad 200 and a flexible hose 210 connecting the housing to the therapy pad. In a preferred embodiment, the housing 110 has a rectangular box shape, but one of ordinary skill in the art will appreciate that the housing may have a different geometric shape, provided that shape does not impair its ability to house the components described herein.

FIG. 2 shows an alternative embodiment of the device. In this embodiment, a fan is used to transfer heat away from the thermoelectric chips. In other words, instead of using fluid to cool down the hot side of the chips, the device employs air flow. Control unit 150 controls the fan 310 and transmits electric current to the thermoelectric chips inside therapy pad 200. In cooling mode, current is supplied to the thermoelectric chips causing one side to cool, i.e. the side next to the user's skin 400, and the opposite side to heat up. The fan is activated to direct heat away from the warm side of the thermoelectric chips, i.e. the side opposite the user's skin 400. In heating mode, the fan is turned off and electric current is reversed. The cool side thus becomes warm, i.e. the side next to the user's skin 400, and the warm side becomes cool. When the fan is deactivated, heat builds up in the thermoelectric chips as in the previously described embodiment. The therapy pad thus radiates heat onto a user's skin 400. In either mode, current is supplied to the thermoelectric chips, but is reversed when changing modes, thereby causing the cool side to turn hot and the hot side to turn cool. A power cord 170 may be used to draw current from a standard wall outlet. In addition, temperature sensors strategically positioned in the device may be used to transmit temperature data to the control unit 150 in order to keep the temperature of the therapy pad within a predefined normal range.

As shown in FIG. 1, the therapy pad 200 includes a plurality of thermoelectric chips 300. The thermoelectric chips may assume a cooling state (cooling mode) or heat generating (heating mode) state depending on whether they are exposed to fluid in fluid line 220. More specifically, in cooling mode a voltage is applied across the chip, and as a result, a difference in temperature will build up between the two sides. The warming side however will be cooled by the fluid line 220 connected to the chip, which acts as a heat sink. The chips thus cause the therapy pad to feel cool and absorb heat from the user skin. In heating mode, fluid is not circulating in the therapy pad (i.e. the pump is turned off) so that heat is allowed to build up on the warming side.

The hose 210 connecting the housing to the therapy pad carries the fluid transmission line 220 therein, which circulates fluid from the reservoir to the therapy pad 200 and back to the reservoir. The fluid line is connected to the pump 140 which pumps fluid through the fluid line 220. The fluid line 220 travels from the pump 140 through hose 210 and enters the therapy pad 200 where the hose connects to the therapy pad. The fluid line 220 winds through the pad 200, and connects to the thermoelectric chips 300. The fluid line then exits the therapy pad as shown in the embodiment of FIG. 1. In an alternate embodiment, the thermoelectric chips are encased in pouches along the fluid line 220 that fill up with fluid when fluid is pumped through the line. The thermoelectric chips are thus immersed in fluid when fluid circulates through the therapy pad and are instantly cooled by the fluid.

The hose 210 also carries a conductor 230, such as an electrical wire, for transmitting an electrical current from the power supply to the thermoelectric chips 300. The controller 150 is configured to turn on/off the electricity. As previously mentioned, one side of the thermoelectric chips heat up when they are exposed to an electrical current. Consequently, when an electrical current is transmitted by the controller to the thermoelectric chips, the thermoelectric chips generate heat which radiates from the therapy pad.

According to an embodiment of the invention, the alternating heat and cold therapy apparatus 100 has a heating mode and a cooling mode. In cooling mode the thermoelectric chips 300 are in a cooling state and are able to provide a cooling sensation through the therapy pad. The apparatus is put into cooling mode when current flows through the thermoelectric chips causing a temperature differential to build up between the two sides of the chips, i.e. one side gets cooler while the other side gets warmer. The hydraulic pump 140 operates to send cooling fluid from the reservoir 120 into the fluid line 220 inside the therapy pad 200 to draw heat away from the warm side of the thermoelectric chips 300. Alternatively, the thermoelectric chips 300 are retained inside pouches along the fluid line 220 and are immersed in fluid when fluid circulates through the line. In this embodiment, the thermoelectric chips are instantly cooled by the fluid. However, in both embodiments, fluid absorbs heat from the thermoelectric chips and carries it away from the therapy pad 200.

In heating mode, the controller 150 also transmits current to the thermoelectric chips, but turns off the hydraulic pump 140. The thermoelectric chips 300 thus receive an electrical charge from the conductor 230 which causes one side to heat up. However, in heating mode heat is allowed to build up instead of being dissipated by a heat sink (e.g. fluid line 220).

According to an embodiment of the invention, the alternating hot and cold therapy apparatus may also include temperature sensors. Sensors may be located in the therapy pad, the connecting hose, or any other area of the apparatus. Temperature sensors in the connecting hose can monitor and relay fluid temperature data to the control unit. Similarly, temperature sensors in the therapy pad can monitor the temperature of the pad and transmit temperature data to the controller. The controller receives data from the temperature sensors and is configured to activate or deactivate heating or cooling mode based on the temperature data. For example, if the temperature of the therapy pad rises above a predetermined maximum threshold, the controller turns off the electrical current and optionally activates the hydraulic pump to circulate cooling fluid until the temperature is back within a predetermined normal range. If, on the other hand, the temperature of the therapy pad is below a minimum threshold, the controller turns off the hydraulic pump and optionally turns off the electrical current applied to the thermoelectric chips until the temperature is back within the normal range.

Turning to FIG. 2, an alternate embodiment of the invention is shown. In cooling mode, a fan 310 serves as a heat sink for the heat generated by the thermoelectric chips in therapy pad 200. In other words, the fan directs heat away from thermoelectric chips in therapy pad 200. Just as in the previous embodiment, a current is applied to the thermoelectric chips inside the therapy pad causing a temperature differential between opposite sides of the chip. Unlike the previous embodiment, air rather than fluid is used as a heat sink to transfer heat away from the thermoelectric chips.

In heating mode, the fan is turned off by the controller 150 and thermoelectric chips in the pad receive an electrical current causing a temperature differential between the opposite sides. However, in heating mode heat is allowed to build up on one side since no heat sink is provided (i.e. the fan is turned off). The controller 150 controls heating and cooling modes by turning on/off the hydraulic pump, fan, or electric power to the thermoelectric chips. As discussed earlier, the controller may be programmed to alternate between heating and cooling modes automatically, or may be manually set to heating or cooling modes.

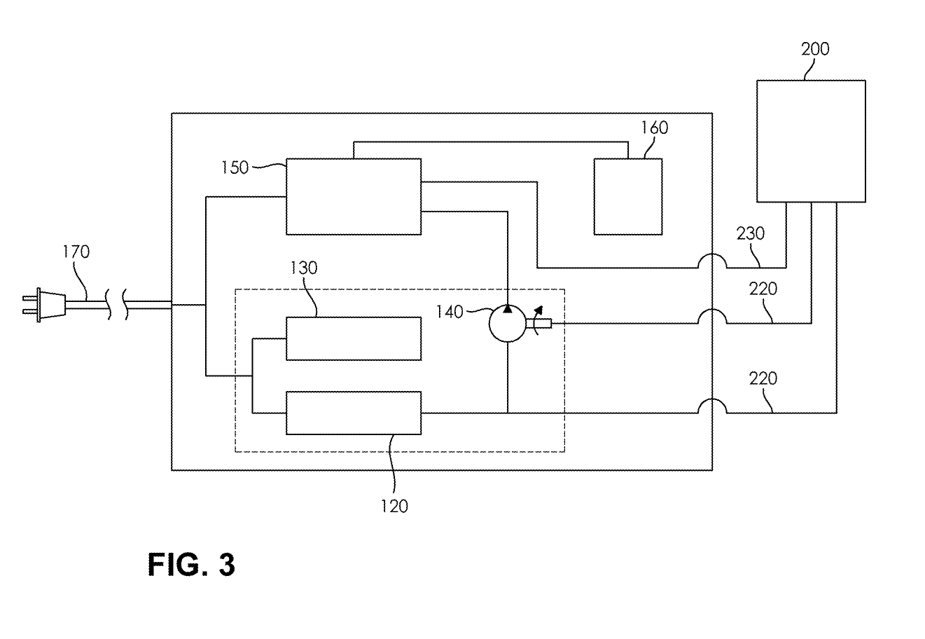

Turning to FIG. 3, an internal schematic of the housing is shown. The schematic shows how each component inside the housing is connected. For example the hydraulic pump 140 is in fluid communication with the fluid reservoir 120. The fluid line 220 is connected to the pump and is carried within hose 210. Hose 210 connects the housing 110 to the therapy pad 200. The controller 150 activates and deactivates the pump and also sends an electrical charge to the thermoelectric chips through conductor 230. Conductor 230 is similarly carried within hose 210.

Hence, both fluid line 220 and conductor 230 are connected to therapy pad 200 via hose 210. A timer 160 provides time data to controller 150 for setting timed intervals of heating and cooling. The timer may also be used by the controller to run programmed heating/cooling sessions. A power supply provides electrical power to the controller, hydraulic pump, and the thermoelectric chips. A power cord 170 is provided to allow the apparatus to be plugged into an electrical outlet.

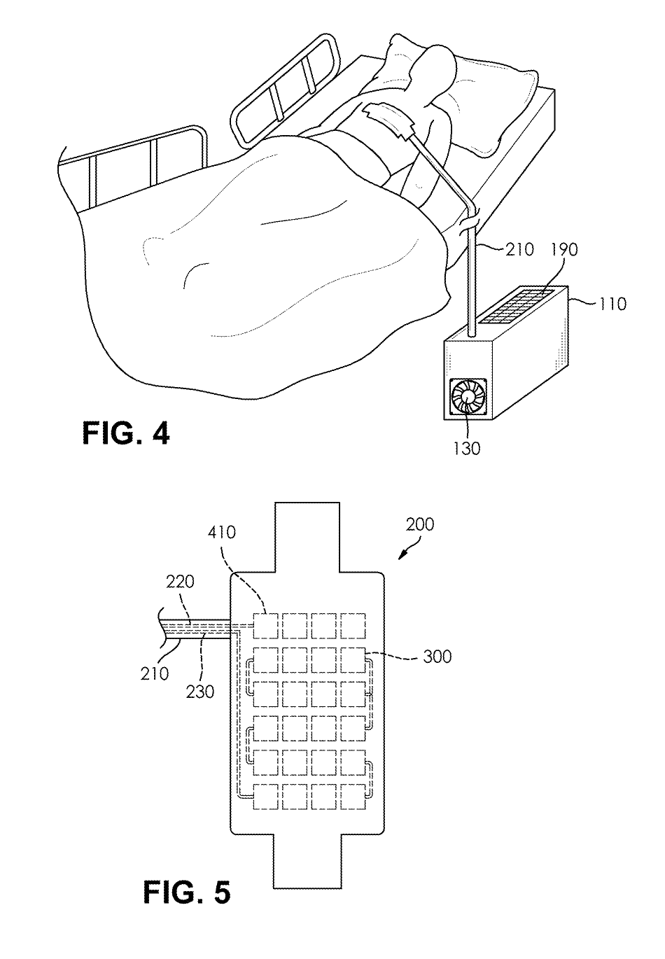

FIG. 4 shows the alternating heat and cold therapy apparatus in use. The housing sits at the side of a user's bed, while the flexible connecting hose is used to position the therapy pad on a pain affected area of the user's body. In this embodiment, the programmable controller can automatically provide alternating heating and cooling according to a preset program. Alternatively, the user may choose to manually control the alternating hot and cold therapy apparatus by manually selecting heating or cooling mode, thus bypassing the preset program.

FIG. 5 shows a schematic view of the therapy pad 200. The thermoelectric chips 300, fluid line and conductor are shown in broken lines to indicate that they are inside the therapy pad and hidden from view unless the therapy pad is opened. The thermoelectric chips 300 are shown in series with the fluid line and conductor winding through the therapy pad in contact with each thermoelectric chip. In cooling mode, this arrangement allows fluid in the fluid line to absorb heat from the thermoelectric chips, or in heating mode exposes the thermoelectric chips to an electrical charge which causes one side to heat up. Since one side of the thermoelectric chips will tend to be cooler or warmer than the other side due to the temperature differential on the chip, one side of the therapy pad will tend to be cooler or warmer. The user can therefore apply the warmer/cooler side of the therapy pad to an affected area, as needed.

While the invention has been thus described with reference to the embodiments, it will be readily understood by those skilled in the art that equivalents may be substituted for the various elements and modifications made without departing from the spirit and scope of the invention. It is to be understood that all technical and scientific terms used in the present invention have the same meaning as commonly understood by one of ordinary skill in the art to which this invention belongs.

1. An alternating heat and cold therapy apparatus comprising:
a housing having
a fluid reservoir inside said housing,
a hydraulic pump in fluid communication with said reservoir,
a programmable controller operably connected to said hydraulic pump, and
a power supply for supplying power to said hydraulic pump and said control unit,
a therapy pad comprising a plurality of thermoelectric chips, wherein said thermoelectric chips generate a temperature differential in response to electric current;
a flexible hose connecting the housing to the therapy pad, said hose containing at least one fluid line connected to said pump, wherein said fluid line travels from said pump through said hose and into said therapy pad and returns to said housing, wherein said fluid line circulates fluid inside the therapy pad when said pump is operating and returns it to said reservoir,
wherein said hose further contains at least one conductor for transmitting an electrical charge from said power supply to said thermoelectric chips, wherein said controller controls transmission of said electrical charge;
wherein the heating and cooling apparatus has a heating mode and a cooling mode,
wherein during cooling mode the thermoelectric chips receive an electric current causing one side of the chips to cool and the other side to warm,
wherein the hydraulic pump operates to send fluid from the reservoir into the fluid line inside the therapy pad, wherein the fluid line is connected to the plurality of thermoelectric chips such that the fluid absorbs heat from the warm side of the thermoelectric chips and carries it away from the therapy pad;
wherein during heating mode, the thermoelectric chips receive an electrical current from the conductor that heats up one side of the chip and the hydraulic pump is turned off;
wherein the controller is configured to activate and deactivate cooling mode and heating mode;
wherein the power supply provides electrical current to the pump, control unit, and thermoelectric chips;
wherein during normal use the controller activates heating mode and cooling mode, so as to provide alternating heating and cooling to an area in contact with the therapy pad.

2. The apparatus of claim 1, wherein said apparatus contains one or more temperature sensors in communication with said controller.

3. The apparatus of claim 2, wherein said controller is configured to activate and deactivate heating or cooling mode based, at least in part, on temperature data received from said sensors.

4. The apparatus of claim 1, wherein said housing comprises a means of cooling the liquid in said reservoir.

5. The apparatus of claim 3, wherein when the temperature of the therapy pad reaches a predetermined maximum threshold, electrical power to the thermoelectric chips is cut off until temperature falls below the threshold, and when the temperature of the therapy pad reaches a predetermined minimum threshold, the pump is turned off and electrical power is transmitted to the thermoelectric chips until the temperature rises above the minimum threshold.

6. The apparatus of claim 1, wherein said housing comprises a vent for releasing heat.

7. The apparatus of claim 4, wherein the means of cooling is a fan.

8. The apparatus of claim 1, wherein the thermoelectric chips are held within pouches inside the fluid line.

9. The apparatus of claim 3, wherein said hose contains one or more temperature sensors.

10. The apparatus of claim 3, wherein said therapy pad contains one or more temperature sensors.

11. The apparatus of claim 1, wherein heating mode or cooling mode may be programmed to continue for a specified duration.

12. The apparatus of claim 1, wherein heating and cooling modes may be programmed to alternate according to one or more specified patterns.

13. The apparatus of claim 1, wherein a user can manually switch between heating mode and cooling mode.

14. An alternating heat and cold therapy apparatus comprising:
a housing having
a fluid reservoir inside said housing,
a hydraulic pump in fluid communication with said reservoir,
a programmable controller operably connected to said hydraulic pump, and
a fan for removing heat from the housing, and
a power supply for supplying power to said hydraulic pump, said fan, and said control unit; and
a therapy pad comprising a plurality of thermoelectric chips, wherein said thermoelectric chips generate a temperature differential in response to electric current;
one or more hoses connecting the housing to the therapy pad, each of said hoses containing at least one fluid line connected to said pump, wherein said fluid line travels from said pump through said one or more hoses and into said therapy pad and returns to said housing, wherein said fluid line circulates fluid inside the therapy pad when said pump is operating and returns it to said reservoir,
wherein said one or more hoses further contains at least one conductor for transmitting an electrical charge from said power supply to said thermoelectric chips, wherein said controller controls transmission of said electrical charge;
wherein the heating and cooling apparatus has a heating mode and a cooling mode,
wherein during cooling mode the thermoelectric chips receive an electric current causing one side of the chips to cool and the other side to warm, wherein the hydraulic pump operates to send fluid from the reservoir into the fluid line inside the therapy pad, wherein the fluid line is connected to the plurality of thermoelectric chips such that the fluid absorbs heat from the warm side of the thermoelectric chips and carries it away from the therapy pad to the reservoir inside the housing, wherein said fan removes heat from inside the housing when turned on;
wherein during heating mode, the thermoelectric chips receive an electrical charge from the conductor that causes one side of the chips to warm up, the hydraulic pump is turned off, and the fan is turned off;
wherein the controller is configured to activate and deactivate cooling mode and heating mode;
wherein the power supply provides electrical power to the pump, control unit, fan, and thermoelectric chips;
wherein during normal use the controller activates heating mode and cooling mode, so as to provide alternating heating and cooling to an area in contact with the therapy pad.

15. The apparatus of claim 14, wherein said apparatus contains one or more temperature sensors in communication with said controller.

16. The apparatus of claim 15, wherein said controller is configured to activate and deactivate heating or cooling mode based, at least in part, on temperature data received from said sensors.

17. The apparatus of claim 16, wherein when the temperature of the therapy pad reaches a predetermined maximum threshold, electrical power to the thermoelectric chips is cut off until temperature falls below the threshold, and when the temperature of the therapy pad reaches a predetermined minimum threshold, the pump is turned off and electrical power is transmitted to the thermoelectric chips until the temperature rises above the minimum threshold.

18. The apparatus of claim 14, wherein heating mode or cooling mode may be programmed to continue for a specified duration.

19. The apparatus of claim 14, wherein heating and cooling modes may be programmed to alternate according to one or more specified patterns.

20. The apparatus of claim 14, wherein a user can manually switch between heating mode and cooling mode.
